SEQUENCE LISTING (78 sequences)

SEQ ID NO: 1 - protein, 13 aa, Artificial Sequence
  Peptide from Threonine--tRNA ligase, cytoplasmic
GAYIYNALIEFIR

SEQ ID NO: 2 - protein, 13 aa, Artificial Sequence
  Peptide from Tubulin alpha-1B chain
  MOD_RES 8: Carbamidomethyl modification
SIQFVDWCPTGFK

SEQ ID NO: 3 - protein, 14 aa, Artificial Sequence
  Peptide from Threonine--tRNA ligase, cytoplasmic
WELNSGDGAFYGPK

SEQ ID NO: 4 - protein, 22 aa, Artificial Sequence
  Peptide from Threonine--tRNA ligase, cytoplasmic
  MOD_RES 16: Carbamidomethyl modification
VNNVVWDLDRPLEEDCTLELLK

SEQ ID NO: 5 - protein, 6 aa, Artificial Sequence
  Peptide from Threonine--tRNA ligase, cytoplasmic
QAEEEF

SEQ ID NO: 6 - protein, 16 aa, Artificial Sequence
  Peptide from Threonine--tRNA ligase, cytoplasmic
NAQLAQYNFILVVGEK

SEQ ID NO: 7 - protein, 25 aa, Artificial Sequence
  Peptide from 26S proteasome non-ATPase regulatory subunit 4
AAAASAAEAGIATTGTEDSDDALLK

SEQ ID NO: 8 - protein, 15 aa, Artificial Sequence
  Peptide from Isoform 2 of T-complex protein 1 subunit theta
AIADTGANVVVTGGK

SEQ ID NO: 9 - protein, 14 aa, Artificial Sequence
  Peptide from Tubulin beta-4B chain
NSSYFVEWIPNNVK

SEQ ID NO: 10 - protein, 10 aa, Artificial Sequence
  Peptide from UV excision repair protein RAD23 homolog B
IDIDPEETVK

SEQ ID NO: 11 - protein, 13 aa, Artificial Sequence
  Peptide from Immunoglobulin-binding protein 1
DSSREASTSNSSR

SEQ ID NO: 12 - protein, 10 aa, Artificial Sequence
  Peptide from Tubulin beta-4B chain
LAVNMVPFPR

SEQ ID NO: 13 - protein, 8 aa, Artificial Sequence
  Peptide from Isoform 2 of Leucine-rich repeat- containing protein 7
TLPKSMHK

SEQ ID NO: 14 - protein, 12 aa, Artificial Sequence
  Peptide from 26S proteasome non-ATPase regulatory subunit 13
ITVNEVELLVMK

SEQ ID NO: 15 - protein, 21 aa, Artificial Sequence
  Peptide from Tubulin alpha-1B chain
FDGALNVDLTEFQTNLVPYPR

SEQ ID NO: 16 - protein, 13 aa, Artificial Sequence
  MOD_RES 1: ACETYLATION
  Peptide from Acylamino-acid-releasing enzyme
  MOD_RES 6: Oxidation
TPLLLMLGQEDRR

SEQ ID NO: 17 - protein, 12 aa, Artificial Sequence
  Peptide from 26S proteasome non-ATPase regulatory subunit 3
SYNKDLESAEER

SEQ ID NO: 18 - protein, 14 aa, Artificial Sequence
  Peptide from Alanine--tRNA ligase, cytoplasmic
  MOD_RES 3,5: Carbamidomethyl modification
ITCLCQVPQNAANR

SEQ ID NO: 19 - protein, 13 aa, Artificial Sequence
  Peptide from 26S proteasome non-ATPase regulatory subunit 14
AVAVVVDPIQSVK

SEQ ID NO: 20 - protein, 23 aa, Artificial Sequence
  Peptide from Isoform 2 of 26S proteasome non- ATPase regulatory subunit 1
TVGTPIASVPGSTNTGTVPGSEK

SEQ ID NO: 21 - protein, 11 aa, Artificial Sequence
  MOD_RES 1,6: METHYLATION
  Peptide from Ubiquitin carboxyl-terminal hydrolase 14
QDEWIKFDDDK

SEQ ID NO: 22 - protein, 14 aa, Artificial Sequence
  Peptide from Isoform 2 of Ubiquitin-like modifier-activating enzyme 1
QPAENVNQYLTDPK

SEQ ID NO: 23 - protein, 8 aa, Artificial Sequence
  Peptide from Ubiquitin-like modifier-activating enzyme 1
NIILGGVK

SEQ ID NO: 24 - protein, 15 aa, Artificial Sequence
  Peptide from Isoform 3 of Calpastatin
DTSQSDKDLDDALDK

SEQ ID NO: 25 - protein, 14 aa, Artificial Sequence
  Peptide from Eukaryotic translation initiation factor 1A, Y-chromosomal
VWINTSDIILVGLR

SEQ ID NO: 26 - protein, 7 aa, Artificial Sequence
  Peptide from Eukaryotic translation initiation factor 1A, Y-chromosomal
YNADEAR

SEQ ID NO: 27 - protein, 11 aa, Artificial Sequence
  MOD_RES 1: ACETYLATION
  Peptide from Splicing factor 3B subunit 1
EFQSPDEEMKK

SEQ ID NO: 28 - protein, 7 aa, Artificial Sequence
  Peptide from Rho GTPase-activating protein 1
DAAITLK

SEQ ID NO: 29 - protein, 13 aa, Artificial Sequence
  Peptide from 26S protease regulatory subunit 7
QTLQSEQPLQVAR

SEQ ID NO: 30 - protein, 18 aa, Artificial Sequence
  Peptide from 26S proteasome non-ATPase regulatory subunit 2
DTYARWLPLGLGLNHLGK

SEQ ID NO: 31 - protein, 5 aa, Artificial Sequence
  Peptide from RuvB-like 2
VAEWR

SEQ ID NO: 32 - protein, 16 aa, Artificial Sequence
  Peptide from Rab3 GTPase-activating protein catalytic subunit
  MOD_RES 6: Carbamidomethyl modification
  MOD_RES 7: Oxidation
LALCLCMINFYHGGLK

SEQ ID NO: 33 - protein, 6 aa, Artificial Sequence
  Peptide from Isoform 2 of 2-oxoglutarate dehydrogenase, mitochondrial
VAAEWR

SEQ ID NO: 34 - protein, 14 aa, Artificial Sequence
  Peptide from Uroporphyrinogen-III synthase
  MOD_RES 8: Carbamidomethyl modification
AVEAAELCLEQNNK

SEQ ID NO: 35 - protein, 11 aa, Artificial Sequence
  MOD_RES 1: ACETYLATION
  Peptide from T-complex protein 1 subunit alpha
MEGPLSVFGDR

SEQ ID NO: 36 - protein, 12 aa, Artificial Sequence
  Peptide from Alanine--tRNA ligase, cytoplasmic
AVFDETYPDPVR

SEQ ID NO: 37 - protein, 14 aa, Artificial Sequence
  Peptide from Isoform 2 of Prothymosin alpha
AAEDDEDDDVDTKK

SEQ ID NO: 38 - protein, 9 aa, Artificial Sequence
  Peptide from Pre-mRNA-processing-splicing factor 8
GYLPSHYER

SEQ ID NO: 39 - protein, 6 aa, Artificial Sequence
  Peptide from 78 kDa glucose-regulated protein
EFFNGK

SEQ ID NO: 40 - protein, 14 aa, Artificial Sequence
  Peptide from Threonine--tRNA ligase, cytoplasmic
GFQEVVTPNIFNSR

SEQ ID NO: 41 - protein, 10 aa, Artificial Sequence
  Peptide from Isoform 2 of CXXC-type zinc finger protein 1
  MOD_RES 2,7: Carbamidomethyl modification
QCNRHYCWEK

SEQ ID NO: 42 - protein, 12 aa, Artificial Sequence
  Peptide from Dystrophin
VAAAETAKHQAK

SEQ ID NO: 43 - protein, 12 aa, Artificial Sequence
  Peptide from Nicotinate phosphoribosyltransferase
  MOD_RES 2,3: METHYLATION
ARDAAEFELFFR

SEQ ID NO: 44 - protein, 8 aa, Artificial Sequence
  Peptide from Farnesyl pyrophosphate synthase
QDADSLQR

SEQ ID NO: 45 - protein, 9 aa, Artificial Sequence
  Peptide from Acylamino-acid-releasing enzyme 4
  MOD_RES 6: Carbamidomethyl modification
LGIRFCTNR

SEQ ID NO: 46 - protein, 15 aa, Artificial Sequence
  Peptide from Isoform 2 of Ubiquitin carboxyl- terminal hydrolase 14
ETDSSSASAATPSKK

SEQ ID NO: 47 - protein, 14 aa, Artificial Sequence
  Peptide from Peptidyl-prolyl cis-trans isomerase FKBP4
SNTAGSQSQVETEA

SEQ ID NO: 48 - protein, 11 aa, Artificial Sequence
  Peptide from Eukaryotic translation initiation factor 1A, Y-chromosomal
EDGQEYAQVIK

SEQ ID NO: 49 - protein, 13 aa, Artificial Sequence
  Peptide from Rab GDP dissociation inhibitor beta
  MOD_RES 12: Oxidation
RMTGSEFDFEEMK

SEQ ID NO: 50 - protein, 9 aa, Artificial Sequence
  Peptide from Hemoglobin subunit gamma-1
MGHFTEEDK

SEQ ID NO: 51 - protein, 10 aa, Artificial Sequence
  Peptide from Drebrin-like protein
NEQESAVHPR

SEQ ID NO: 52 - protein, 16 aa, Artificial Sequence
  Peptide from Src substrate cortactin
TQTPPVSPAPQPTEER

SEQ ID NO: 53 - protein, 5 aa, Artificial Sequence
  Peptide from Abnormal spindle-like microcephaly-associated protein
LLDLR

SEQ ID NO: 54 - protein, 8 aa, Artificial Sequence
  Peptide from GTP-binding nuclear protein Ran
HLTGEFEK

SEQ ID NO: 55 - protein, 18 aa, Artificial Sequence
  Peptide from Protein Hook homolog 1
KYFHAQLQLEQLQEENFR

SEQ ID NO: 56 - protein, 6 aa, Artificial Sequence
  Peptide from Protein phosphatase methylesterase 1
SMHLGR

SEQ ID NO: 57 - protein, 11 aa, Artificial Sequence
  Peptide from Tropomyosin alpha-3 chain
  MOD_RES 10: METHYLATION
QLEDELAAMQK

SEQ ID NO: 58 - protein, 19 aa, Artificial Sequence
  MOD_RES 1,13: METHYLATION
  Peptide from Selenium-binding protein 1
CGNCGPGYSTPLEAMKGPR

SEQ ID NO: 59 - protein, 8 aa, Artificial Sequence
  MOD_RES 1: ACETYLATION
  Peptide from Isoform 2 of F-actin-capping protein subunit beta
SVQTFADK

SEQ ID NO: 60 - protein, 7 aa, Artificial Sequence
  Peptide from Isoform 2 of 26S protease regulatory subunit 8
FVVDVDK

SEQ ID NO: 61 - protein, 10 aa, Artificial Sequence
  Peptide from Isoform 2 of DnaJ homolog subfamily B member 2
REQQPSVTSR

SEQ ID NO: 62 - protein, 10 aa, Artificial Sequence
  Peptide from LIM and SH3 domain protein 1
  MOD_RES 5: Carbamidomethyl modification
MNPNCARCGK

SEQ ID NO: 63 - protein, 17 aa, Artificial Sequence
  Peptide from Threonine--tRNA ligase, cytoplasmic
  MOD_RES 11: Carbamidomethyl modification
QVMVVPVGPTCDEYAQK

SEQ ID NO: 64 - protein, 16 aa, Artificial Sequence
  Peptide from 28 kDa heat- and acid-stable phosphoprotein
QYTSPEEIDAQLQAEK

SEQ ID NO: 65 - protein, 9 aa, Artificial Sequence
  Peptide from Putative G antigen family E member 3
VLEAGDAQP

SEQ ID NO: 66 - protein, 15 aa, Artificial Sequence
  Peptide from Isoform 4 of Golgin subfamily B member 1
AEEADHEVLDQKEMK

SEQ ID NO: 67 - protein, 15 aa, Artificial Sequence
  MOD_RES 1: Glycation
  Peptide from Putative protein FAM10A5 1
MCKQDPSVLHTEEMR

SEQ ID NO: 68 - protein, 13 aa, Artificial Sequence
  MOD_RES 1: ACETYLATION
  Peptide from 26S protease regulatory subunit 4
YDSNSGGEREIQR

SEQ ID NO: 69 - protein, 16 aa, Artificial Sequence
  Peptide from Eukaryotic translation initiation factor 1A, Y-chromosomal
ELVFKEDGQEYAQVIK

SEQ ID NO: 70 - protein, 28 aa, Artificial Sequence
  Peptide from Isoform 2 of V-type proton ATPase subunit F
SLGSLPGSVVEANPNQRDPPLWDEIDSR

SEQ ID NO: 71 - protein, 21 aa, Artificial Sequence
  Peptide from Platelet-activating factor acetylhydrolase IB subunit alpha
  MOD_RES 8: Oxidation
MWDVSTGMCLMTLVGHDNWVR

SEQ ID NO: 72 - protein, 9 aa, Artificial Sequence
  Peptide from T-complex protein 1 subunit beta
VAEIEHAEK

SEQ ID NO: 73 - protein, 16 aa, Artificial Sequence
  Peptide from Isoform SV of 14-3-3 protein epsilon
GDYHRYLAEFATGNDR

SEQ ID NO: 74 - protein, 20 aa, Artificial Sequence
  Peptide from Threonine--tRNA ligase, cytoplasmic
  MOD_RES 15: Carbamidomethyl modification
DQELYFFHELSPGSCFFLPK

SEQ ID NO: 75 - protein, 11 aa, Artificial Sequence
  Peptide from T-complex protein 1 subunit alpha
IHPTSVISGYR

SEQ ID NO: 76 - protein, 8 aa, Artificial Sequence
  Peptide from T-complex protein 1 subunit beta
LAVEAVLR

SEQ ID NO: 77 - protein, 8 aa, Artificial Sequence
  Peptide from Protein CDV3 homolog
SLDNFFAK

SEQ ID NO: 78 - protein, 13 aa, Artificial Sequence
  Peptide from Casein kinase II subunit alpha
GGPNIITLADIVK

(19) United States
(12) Patent Application Publication
(10) Pub. No.: US 2020/0400693 A1
(43) Pub. Date: Dec. 24, 2020

(54) METHODS FOR THE DETECTION OF AUTOLOGOUS BLOOD-DOPING

(71) Applicant: PRO TEST DIAGNOSTICS AB, Umeå (SE)

(72) Inventors: Christer MALM, Vännäs (SE); Nelson KHOO, Holmsund (SE); Irene GRANLUND, Umeå (SE); Pernilla LINDÉN, Umeå (SE); Michael HALL, Umeå (SE); Raik WAGNER, Umeå (SE)

(21) Appl. No.: 16/976,936

(22) PCT Filed: Mar. 1, 2019
(86) PCT No.: PCT/EP2019/055209

§ 371 Date: Aug. 31, 2020

(30) Foreign Application Priority Data
Mar. 1, 2018 (SE) 1830069-9

(51) Int. Cl. G01N 33/80

(52) U.S. Cl. CPC G01N 33/80; G01N 2560/00; C12Y 304/21004

(57) ABSTRACT

The present invention relates to the identification of peptides, and the corresponding proteins, that can be used in methods for the detection of autologous blood doping. More specifically, the invention relates to methods comprising tryptic digestion of samples of isolated red blood cell (RBC), specifically isolated RBC cytosol, followed by peptide mapping using liquid chromatography tandem-mass spectroscopy (LC-MS/MS). The methods according to the invention which enable detection of increased levels of certain peptides in samples from subjects that have been subjected to autologous blood doping, compared to samples from non-doped control subjects.

The present invention relates to the identification of peptides, and the corresponding proteins, that can be used in methods for the detection of autologous blood doping. More specifically, the invention relates to methods comprising tryptic digestion of samples of isolated red blood cells (RBC), specifically isolated RBC cytosol samples, followed by peptide mapping using liquid chromatography tandem-mass spectroscopy (LC-MS/MS). These methods enable detection of increased levels of certain peptides in samples from subjects that have been subjected to autologous blood doping, compared to samples from non-doped control subjects.

In endurance sports, maximal oxygen uptake (VO₂ₘₐₓ) is an important factor for performance [Ekblom et al. Effect of changes in arterial oxygen content on circulation and physical performance. J Appl Physiol. 1975; 39(1):71-5; Carlsson et al. Oxygen uptake at different intensities and sub-techniques predicts sprint performance in elite male cross-country skiers. Eur J Appl Physiol. 2014; 114(12):2587-95]. Limitations in VO₂ₘₐₓ are, assuming normal lung function and sea-level oxygen tension, maximal cardiac output (Qₘₐₓ) [Grimby et al. Cardiac output during submaximal and maximal exercise in active middle-aged athletes. J Appl Physiol. 1966; 21(4):1150-6.], oxygen carrying capacity of the blood [Ekblom et al. Central circulation during exercise after venesection and reinfusion of red blood cells. J Appl Physiol. 1976; 40(3):379-83; Buick et al. Effect of induced erythrocythemia on aerobic work capacity. J Appl Physiol Resp Environ Exerc Physiol. 1980; 48(4):636-42] and total hemoglobin mass [Schmidt & Pommer. Impact of alterations in total hemoglobin mass on VO2max. Exerc Sport Sci Rev. 2010; 38(2):68-75.]. Extraction of available oxygen to working muscle is also a factor, at least in elite athletes [Bangsbo et al. Muscle oxygen kinetics at onset of intense dynamic exercise in humans. Am J Physiol Regul lntegr Comp Physiol. 2000; 279(3):R899-906].

Depending on mode and duration of work being performed, and the mode of testing, the influence of VO₂ₘₐₓ on physical performance has been found to range from 62% to 88% in cross country skiing [Carlsson et al. ibid] and 42% to 79% in firefighting [Lindberg et al. Field tests for evaluating the aerobic work capacity of firefighters. PLoS One. 2013; 8(7):e68047]. Similar models have previously been reached for running [Tanaka & Matsuura. A multivariate analysis of the role of certain anthropometric and physiological attributes in distance running. Ann Hum Biol. 1982; 9(5):473-82; Farrell et al. Plasma lactate accumulation and distance running performance. Med Sci Sports. 1979; 11(4):338-44.], orienteering [Knowlton et al. Physiological and performance characteristics of United States championship class orienteers. Med Sci Sports Exerc. 1980; 12(3):164-9], cycling [Lamberts & Davidowitz. Allometric scaling and predicting cycling performance in (well-) trained female cyclists. Int J Sports Med. 2014; 35(3):217-22; Coyle et al. Physiological and biomechanical factors associated with elite endurance cycling performance. Med Sci Sports Exerc. 1991; 23(1):93-107], swimming [Chatard et al. Swimming skill and stroking characteristics of front crawl swimmers. Int J Sports Med. 1990; 11(2):156-61; Duche et al. Analysis of performance of prepubertal swimmers assessed from anthropometric and bio-energetic characteristics. Eur J Appl Physiol Occup Physiol. 1993; 66(5):467-71] and triathlon [Barrero et al. Intensity profile during an ultra-endurance triathlon in relation to testing and performance. Int J Sports Med. 2014; 35(14):1170-8].

In intermittent exercise, such as soccer, the influence of VO₂ₘₐₓ on performance is not known, possibly due to difficulties in measuring the dependent outcome variable, i.e. performance, in team sports. Consequently, different methods to enhancing oxygen delivery can be used by cheating athletes, and the effects on physical performance can be substantial [Segura et al. Detection methods for autologous blood doping. Drug Test Anal. 2012; 4(11):876-81].

Initially, blood transfusion was used to enhance military aviation pilots' work capacity to fly at high altitude during World War II, when pressurized cockpits were not used [Pace et al. The increase in hypoxic tolerance of normal men accompanying the polycythaemia induced by transfusion of erythrocytes. Am J Physiol. 1947, 148, 152-63]. Later, submaximal [Robinson et al. Circulatory effects of acute expansion of blood volume: studies during maximal exercise and at rest. Circulation Research. 1966; 19(1):26-32] and maximal [Ekblom et al. Response to exercise after blood loss and reinfusion. J Appl Physiol. 1972; 33(2):175-80] running performance was shown to improve with blood transfusion.

The discovery of erythropoietin (EPO) [Miyake et al. Purification of human erythropoietin. J Biol Chem. 1977; 252(15):5558-64] simplified blood doping in sports, supplementing blood donation, storage and subsequent re-infusion. Similar performance enhancements of 6-12% could now be achieved by a simple recombinant human (rh) EPO injection [Berglund & Ekblom. Effect of recombinant human erythropoietin treatment on blood pressure and some haematological parameters in healthy men. J Intern Med. 1991; 229(2):125-30; Ekblom. Blood boosting and sport. Baillieres Best Pract Res Clin Endocrinol Metab. 2000; 14(1):89-98; Thomsen et al. Prolonged administration of recombinant human erythropoietin increases submaximal performance more than maximal aerobic capacity. Eur J Appl Physiol. 2007; 101(4):481-6]. In a review on blood doping published in 1989, Jones and Tunstall [Blood doping—a literature review. Br J Sports Med. 1989; 23(2):84-8] describe increases in performance and VO₂ₘₐₓ ranging between 0% and 40%, depending on the subjects included and methods used for both testing and doping. From the summarized literature, it can be estimated that elite athletes may improve performance by up to 3% with blood doping, regardless of method [Birkeland et al. Effect of rhEPO administration on serum levels of sTfR and cycling performance. Med Sci Sports Exerc. 2000; 32(7):1238-43; Berglund & Hemmingson. Effect of reinfusion of autologous blood on exercise performance in crosscountry skiers. Int J Sports Med. 1987; 8(3):231-3; Brien & Simon. The effects of red blood cell infusion on 10-km race time. JAMA. 1987; 257(20):2761-5]. This enhancement is equivalent to, for example, a seven minutes faster winning time in the 90 km cross country ski race Vasaloppet, a 20-30 seconds faster time in any given 5000 meter long distance run at world class level, and a four minutes faster finishing time in a marathon race. In cycling, a 3% increase in performance translates to a more than two hour faster winning time in the Tour de France (2014 edition).

The World Anti-Doping Agency (WADA) has banned the use of many techniques to increase the oxygen carrying capacity of blood, including; blood transfusion, hormone injections, artificial oxygen carriers, allosteric Hb modulators and genetic manipulations. While methods to detect rhEPO [Wide et al. Detection in blood and urine of recombinant erythropoietin administered to healthy men. Med Sci Sports Exerc. 1995; 27(11):1569-76; Lasne & de Ceaurriz. Recombinant erythropoietin in urine. Nature. 2000; 405(6787):635] and homologous blood transfusion [Nelson et al. Proof of homologous blood transfusion through quantification of blood group antigens. Haematologica. 2003; 88(11):1284-95] have successfully been developed, no direct method is available for autologous blood transfusion [Pialoux et al. Hemoglobin and hematocrit are not such good candidates to detect autologous blood doping. Int J Hematol. 2009; 89(5):714-5; Jelkmann & Lundby. Blood doping and its detection. Blood. 2011; 118(9):2395-404].

Currently the Athlete Biological Passport (ABP) [Berglund. Development of techniques for the detection of blood doping in sport. Sports Med. 1988; 5(2):127-35; Berglund et al. The Swedish Blood Pass project. Scand J Med Sci Sports. 2007; 17(3):292-7] is the best practice, although with known limitations [Bejder et al. Acute hyperhydration reduces athlete biological passport OFF-hr score. Scand J Med Sci Sports. 2016; 26(3):338-47]. It can therefore be assumed that cheating athletes have returned to the practice of blood transfusions.

One study [Damsgaard et al. Effects of blood withdrawal and reinfusion on biomarkers of erythropoiesis in humans: Implications for anti-doping strategies. Haematologica. 2006; 91(7):1006-8.] investigated the sensitivity of doping detection based on measurements of hemoglobin, hematocrit, reticulocyte percentage (% ret), serum EPO levels, and soluble transferrin receptor. The results showed a significant increase in % ret and decrease in [Hb] as a consequence of the donation. However, donation time is difficult to predict, since cryopreservation makes it possible to store blood for an extended time (years, even decades) [Henkelman et al. Utilization and quality of cryopreserved red blood cells in transfusion medicine. Vox Sanguinis. 2015 108, 103-112]. Hematological [Berglund etal. Effects of blood transfusions on some hematological variables in endurance athletes. Med Sci Sports Exerc. 1989; 21(6):637-42] as well as performance enhancing effects may last for weeks to months in both men [Buick et al. Effect of induced erythrocythemia on aerobic work capacity. J Appl Physiol Resp Environ Exerc Physiol. 1980; 48(4):636-42] and women [Robertson et al. Hemoglobin concentration and aerobic work capacity in women following induced erythrocythemia. J Appl Physiol. 1984; 57(2):568-75]. Thus, sampling blood from any athlete with intention to detect a blood transfusion is subject to chance, when both the donation and re-infusion can be done at any time, including out-of-competition.

A study by Nikolovski et al. [Alterations of the erythrocyte membrane proteome and cytoskeleton network during storage—a possible tool to identify autologous blood transfusion”. 2012. Drug Testing and Analysis 4:882-890] identified that during cold storage red blood cells undergo structural changes that progress over time and can therefore be indicative of both storage per se and length of storage of a blood sample. Cold-storage is not, however, appropriate in the case of autologous blood doping. The donation of blood must be made at a sufficiently long time before re-infusion to ensure that the physiological negative effect of the donation as such is overcome; i.e. the subject has regenerated the lost blood volume and constituents (such as red blood cells) to give an overall positive effect of the re-infusion. This is typically at least 4 weeks [Ekblom et al. Response to exercise after blood loss and reinfusion. J Appl Physiol. 1972; 33(2):175-80]. To minimise the negative effects of donation on continued physical training it may be advantageous to donate smaller volumes of blood, i.e. less than 400 ml, at multiple times during an extended period of time. This need for a length of storage of at least 4 weeks rules out the use of cold-storage, and instead cryopreservation and freeze-storage of the donated blood is required.

An extensive review of various methods to detect autologous blood doping was recently published [Morkeberg J. Detection of autologous blood transfusions in athletes: a historical perspective. Transfusion medicine reviews. 2012; 26(3):199-208]. Recently Maim et al. [Autologous Doping with Cryopreserved Red Blood Cells—Effects on Physical Performance and Detection by Multivariate Statistics. 2016. PLoS ONE 11(6): e0156157] presented a study where recreational male and female athletes were subjected to autologous blood doping in two different transfusion settings. Hematological variables and physical performance were measured before donation of 450 mL or 900 mL cryopreserved whole blood, and until four weeks after re-infusion of the cryopreserved RBC fraction. Significant increase in performance (15±8%) and VO₂ₘₐₓ (17±10%) could be measured 48 h after RBC re-infusion and remained increased for up to four weeks in some subjects. However, hematological variables were found to be inadequate for detection of autologous blood doping.

### DESCRIPTION OF THE INVENTION

The present inventors have now developed a method comprising tryptic digestion of samples of isolated red blood cells (RBC), specifically isolated RBC cytosol samples, followed by peptide mapping using liquid chromatography tandem-mass spectroscopy (LC-MS/MS). The methods according to the invention enable detection of increased levels of certain peptides in samples from subjects that have been subjected to autologous blood doping, compared to samples from non-doped control subjects.

These peptides, and the related proteins, have, in themselves, utility as biomarkers for the detection of autologous blood-doping.

As discussed above, autologous blood doping requires storage of donated blood for a period of time that requires the use of cryopreservation and freeze storage. The present invention is therefore aimed at detecting changes that occur in the red blood cell proteins during the freeze-thawing cycle and using them to identify samples that have undergone that process, including identifying blood samples from subjects that have undergone autologous blood doping.

The inventors have identified that levels of individual tryptic peptides generated during the proteolytic digestion are different between doped and non-doped blood (as seen, for example, in LC/MS-MS measurements after digestion). Without wishing to be bound by theory, the inventors believe that the cryopreservation, freeze storage and/or thawing process may cause structural changes in certain proteins in the red blood cells, for example red blood cell cytosolic proteins. These structural changes appear to remain even after re-infusion and so appear to alter the accessibility of certain cleavage sites for proteolytic enzymes, such as accessibility to certain trypsin sites. This in turn can create the changes in levels of individual tryptic peptides generated during the proteolytic digestion. Therefore, the changes in the levels of certain peptides in the tryptic map is indicative of changes in the structure of the related proteins.

Accordingly, the present invention provides methods for the detection of autologous blood-doping in a subject, said method comprising detection of differences in the levels (also referred to as the amounts) of peptides obtained from blood samples, including red blood cells, following generation of a proteolytic peptide map of red blood cell proteins, preferably red blood cell cytosolic proteins.

In one aspect of the invention there is provided a method for detection of autologous blood-doping in a subject, said method comprising the step:
i) identifying whether said subject has or has not been autologous blood doped based on differences in level of one or more specific peptides between a blood sample from said subject compared to the level of the same specific peptides from a reference blood sample, such levels having been determined by generation of a proteolytic peptide map for each of the blood sample and the reference blood sample.

Peptide mapping of biological samples has been described in numerous publications including, for example in [Saraswathy et al. Protein Identification by Peptide Mass Fingerprinting. In Concepts and Techniques in Genomics and Proteomics, 2011. Woodhead Publishing Ltd.; and in Cottrell, Protein identification using MS/MS data. J Proteomics 2011. 74(10), 1842-1851.]

In one option the method may further comprise performing, before step (i), one or more of the steps of
x) determining the level of one or more specific peptide in the blood sample obtained from said subject;
y) determining the level of one or more specific peptide in a reference blood sample;
z) determining any difference in level of said one or more specific peptides compared to the level of the same specific peptides in a reference blood sample.

The method may comprise the following steps:
a) generating a proteolytic peptide map of a blood sample obtained from said subject;
b) determining the levels of one or more specific peptide identified in the peptide map obtained in step a);
c) determining the difference in level of said one or more specific peptides compared to the level of the same specific peptides in reference peptide maps obtained from a reference blood sample; and
d) identifying whether said subject has or has not been autologous blood doped based on differences in the level of said specific peptides compared to the level of the same specific peptides in said reference peptide maps.

Preferably, the reference blood sample is from a non-doped subject. Preferably, reference blood samples are collected from multiple non-doped subjects of different age, gender and ethnicity, both athletes and non-athletes.

In another aspect of the invention provides methods for detection of autologous blood-doping in a subject, said method comprising the steps:
a) generating a proteolytic peptide map of a blood sample obtained from said subject;
b) determining the level of one or more specific peptides identified in the peptide map obtained in step a);
c) determining the difference in level of said one or more specific peptides compared to the level of the same specific peptides in reference peptide maps obtained from a reference population of non-doped subjects; and
c) identifying said subject as being autologous blood doped based on differences in the level of said specific peptides compared to the level of the same peptides in said reference peptide maps.

In a further aspect of the invention there is provided a method for determining the difference in level of one or more specific peptides in a blood sample from a subject, the method comprising:
a) generating a proteolytic peptide map of the blood sample obtained from said subject;
b) determining or measuring the levels of one or more specific peptide identified in the peptide map obtained in step a); and,
c) determining and/or reporting the difference in level of said one or more specific peptides compared to the level of the same specific peptides in reference peptide maps obtained from a reference population of subjects.

In a yet further aspect of the invention, there is provided a method for determining and/or reporting the difference in level of one or more specific peptides in a blood sample from a subject, the method comprising: comparing the levels of one or more specific peptides in a peptide map generated by proteolytic digestion of the blood sample obtained from said subject, to the level of the same specific peptides in reference peptide maps obtained from a reference population of subjects, thereby determining and/or reporting the difference in level of said one or more specific peptides.

In an alternative aspect of the invention there is provided a method for detecting autologous blood-doping in a subject, said method comprising:
a) generating a proteolytic peptide map of red blood cells (RBCs) or cytosol thereof, wherein the RBCs are isolated from a blood sample obtained from said subject;
b) determining the levels of one or more specific peptide identified in the peptide map obtained in step a);
c) determining the difference in level of said one or more specific peptides compared to the level of the same specific peptides in reference peptide maps obtained from a reference population of non-doped subjects; and,
d) reporting said subject as being autologous blood doped based on differences in the level of said specific peptides compared to the level of the same specific peptides in said reference peptide maps.

In a yet further aspect of the invention there is provided a method for detecting autologous blood-doping in a subject, said method comprising:
a) isolating red blood cells (RBCs) or cytosol thereof, from a blood sample obtained from said subject;
b) generating a proteolytic peptide map of said RBCs or cytosol thereof;
c) determining and/or measuring the levels of one or more specific peptide identified in the peptide map obtained in step a), for ascertaining the difference in level of said one or more specific peptides compared to the level of the same specific peptides in reference peptide maps obtained from a reference population of subjects; and,
d) reporting said subject as being autologous blood doped based on differences in the level of said specific peptides compared to the level of the same specific peptides in said reference peptide maps.

In the aspects of the invention described above, one or more of the following embodiments may apply.

In one embodiment, the reference blood sample is from a non-doped subject. Preferably, reference blood samples are collected from multiple non-doped subjects of different age, gender and ethnicity, both athletes and non-athletes.

In one embodiment, the difference in level of each said specific peptide compared to the level of the same peptide in said reference peptide maps can be more than 10%, such as more than 20%, 30%, 40%, 50%, such as preferably more than 60%, 70%, 80%, 90%, even more preferably more than 100%, 150%, or even more preferably than 200%. The amount of an increase can be can be more than 10%, such as more than 20%, 30%, 40%, 50%, such as preferably more than 60%, 70%, 80%, 90%, even more preferably more than 100%, 150%, or even more preferably than 200%. The amount of a decrease can be more than 10%, such as more than 20%, 30%, 40%, 50%, such as preferably more than 60%, 70%, 80%, 90%, up to an including a 100% decrease i.e. a complete absence of the peptide.

In one embodiment said specific peptides can be one or more peptides derived from one or more of the proteins listed in Table 3.

In one embodiment said specific peptides can be one or more peptides selected from the list of peptides comprising the peptides SEQ ID Nos: 1-78.

Said specific peptides can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, or 78 peptides selected from the list of peptides comprising SEQ ID Nos: 1-78.

In one preferred embodiment said specific peptides can be one or more peptides selected from the list of peptides comprising SEQ ID Nos: 1, 2, 7, 8, 9, 10, 12, 14, 15, 19, 22, 23, 24, 31, 34, 51, 77, 78.

Preferably, said specific peptides can be 1, 2, 3, 4, 5, 6, 7, 8, 9, 10, 11, 12, 13, 14, 15, 16, 17, or 18 peptides from the list of peptides SEQ ID Nos: 1, 2, 7, 8, 9, 10, 12, 14, 15, 19, 22, 23, 24, 31, 34, 51, 77, 78.

In one preferred embodiment said specific peptides can be one or more peptides selected from the list of peptides comprising SEQ ID Nos: 1, 7, 8, 9, 12, 14, 22, 24, 78.

Preferably, said specific peptides can be 1, 2, 3, 4, 5, 6, 7, 8, or 9 peptides from the list of peptides SEQ ID Nos: 1, 7, 8, 9, 12, 14, 22, 24, 78.

Preferably, said specific peptides can be 5 to 10 peptides selected from the list of peptides comprising SEQ ID Nos: 1-78, or 5 to 10 peptides selected from the list of peptides comprising SEQ ID Nos: 1, 2, 7, 8, 9, 10, 12, 14, 15, 19, 22, 23, 24, 31, 34, 51, 77, 78, or 5 to 9 peptides selected from the list of peptides comprising SEQ ID Nos: 1, 7, 8, 9, 12, 14, 22, 24, 78.

Preferably the blood sample to be analyzed in the methods according to the invention is a red blood cell sample obtained from the subject to be tested. Even more preferably the blood sample to be analyzed is an isolated red blood cell cytosol sample obtained from the subject to be tested.

Accordingly, in one embodiment the methods according to the invention can comprise the step of isolating red blood cells from a blood sample obtained from the subject to be tested, prior to the step comprising protease digestion of the obtained red blood cells. Isolation of red blood cells can be performed using any laboratory technique for such isolation, including centrifugation.

Accordingly, in another embodiment the methods according to the invention can comprise the step of isolating red blood cells from a blood sample obtained from the subject to be tested, and a further step comprising isolation of the red blood cell cytosolic fraction, prior to the step comprising protease digestion of the obtained red blood cytosol. The red blood cell cytosolic fraction can be prepared by hypotonic lysis of red blood cells and subsequent removal of the red blood cell membranes by centrifugation.

Accordingly, in another embodiment the methods according to the invention can comprise the step of isolating red blood cells from a blood sample obtained from the subject to be tested, and a further step comprising isolation of the red blood cell cytosolic fraction, and yet another step comprising depletion of the cytosolic fraction of hemoglobin, prior to the step comprising protease digestion of the obtained red blood cytosol.

The blood sample, the isolated red blood sample, or the isolated red blood cell cytosol sample to be tested can further be supplemented with known amounts of one or more reference protein or reference peptide. One or more reference peptides can contain one or more amino acids containing a stable heavy isotope label providing the labelled peptide with a defined increase in molecular weight. The isotope label can be ¹³C, or ¹⁵N.

The reference peptides can be one or more of the peptides selected from the list of peptides comprising SEQ ID Nos: 1-78.

Preferably the proteolytic peptide map is a tryptic peptide map, i.e. the proteolytic digestion of the sample to be tested is performed by trypsin digestion. The proteolytic digestion can also be performed by using one or more of the proteases selected from trypsin, chymotrypsin, Lys-C, Gly-C, Asp-N, Arg-C, papain. [Saraswathy et al., 2011. (supra)]

The peptide mapping can be performed by the combination of liquid chromatography (LC) with mass spectrometry (MS), preferably the MS is tandem mass spectrometry (MS/MS). The liquid chromatography can be high-performance liquid chromatography (HPLC), also known as high pressure liquid chromatography, ultra performance (pressure) liquid chromatography (UPLC), or ultra-high performance (pressure) liquid chromatography (UHPLC). [Fekete et al. Current and future trends in UHPLC. Trends Anal Chem 2014. 63, 2-13].

One aspect of the invention provides an isolated peptide selected from the list of peptides comprising the peptides SEQ ID Nos. 1-35, and 37-78.

Preferably the isolated peptide is selected from the list of peptides comprising the peptides SEQ ID Nos: 1, 2, 7, 8, 9, 10, 12, 14, 15, 19, 22, 23, 24, 31, 34, 51, 77, and 78.

One aspect of the invention provides a kit comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 70, 71, 71, 73, 74, 75, 76, 77 or 78 peptides from the list of peptides SEQ ID Nos: 1-78.

One aspect of the invention provides a kit comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 70, 71, 71, 73, 74, 75, 76, 77, or 78 of the peptides from the list of peptides SEQ ID Nos: 1-78 for use in a method according to the invention.

Preferably the kits of the invention have one or more of the following optional features:
(1) the peptides are stored in individual containers;
(2) combinations of peptides are stored in individual containers;
(3) the peptides are stored in solution;
(4) the peptides are lyophilized;
(5) the peptides are provided in an amount of 450 fmol up to 60000 fmol;
(6) the peptides are, when stored in solution, provided in a concentration of 0.025 μM to 10000 μM.

The kit may further comprise peptides of non-human origin for quality and performance assurance, e.g. peptides with proteolytic sites for performance control of the proteolytic step.

Another aspect of the invention provides a method for the identification of biomarkers for the detection of autologous blood-doping, said method comprising the steps;
i) identifying differences in the level of one or more peptides between blood samples obtained from one or more subjects having received an infusion of autologous blood and from blood samples obtained from one or more control subjects not having received an infusion of autologous blood, such levels having been identified following generation a proteolytic peptide map of the blood samples; and
ii) identifying peptides being present in significant different levels, as a biomarker for autologous blood doping.

Preferably, the proteolytic peptide map is generated from red blood cells obtained from the blood sample(s).

Such a method may comprise the steps;
i) generating proteolytic peptide maps of red blood cells prepared from blood samples obtained from one or more individuals having received an infusion of autologous blood,
ii) generating proteolytic peptide maps of red blood cells prepared from blood samples obtained from one or more control individuals not having received an infusion of autologous blood,
iii) identifying differences in the level of one or more peptides in the peptide maps obtained in step i) compared to peptide maps obtained in step ii), and
iv) identifying peptides being present in significant different levels, and the corresponding proteins, as biomarker for autologous blood doping.

In an alternative aspect of the invention, there is provided a method for identifying proteins having modified expression level post autologous blood-doping, said method comprising: comparing the level of one or more peptides in peptide maps generated by proteolytic digestion of red blood cells (RBCs) or cytosol thereof prepared from blood samples obtained from one or more individuals having received an infusion of autologous blood, to the level of the same specific peptides in reference peptide maps generated by proteolytic digestion of red blood cells (RBCs) or cytosol thereof prepared from blood samples obtained from one or more individuals not having received an infusion of autologous blood, thereby identifying peptides having modified expression level post autologous blood-doping.

Such method may alternatively comprise the steps:
i) generating proteolytic peptide maps of red blood cells (RBCs) or cytosol thereof prepared from blood samples obtained from one or more individuals having received an infusion of autologous blood,
ii) generating proteolytic peptide maps of RBCs or cytosol thereof prepared from blood samples obtained from one or more control individuals not having received an infusion of autologous blood,
iii) identifying differences in the level of one or more peptides in the peptide maps obtained in step i) compared to peptide maps obtained in step ii), and
iv) identifying peptides being present in significant different levels, thereby identifying the corresponding proteins as having modified expression level post autologous blood-doping.

The above two aspects of the invention may include one or more of the following embodiments.

Preferably the red blood sample to be analyzed is an isolated red blood cell cytosol sample.

Preferably the cytosol sample has been depleted of hemoglobin or has a reduced level of hemoglobin.

Preferably the proteolytic peptide map is a tryptic peptide map, i.e. the proteolytic digestion of the proteins is performed by trypsin digestion. The proteolytic digestion can also be performed by using one or more of the proteases selected from trypsin, chymotrypsin, Lys-C, Gly-C, Asp-N, Arg-C, papain.

The peptide mapping can be performed by the combination of liquid chromatography (LC) with mass spectrometry (MS), preferably the MS is tandem mass spectrometry (MS/MS). The liquid chromatography can be high-performance liquid chromatography (HPLC), also known as high pressure liquid chromatography, ultra performance (pressure) liquid chromatography (UPLC), or ultra-high performance (pressure) liquid chromatography (UHPLC).

The kits and methods of the invention as described in the various aspects above may include or can allow for the identification of peptides of interest, whereby samples of known peptides allow the testing lab to positively identify the corresponding peptides in the sample from the subject and the control sample.

Therefore, the methods may comprise the additional step of identifying peptides of interest by comparison to reference peptides.

In one embodiment, such identification can be performed by the combination of liquid chromatography (LC) with mass spectrometry (MS), preferably the MS is tandem mass spectrometry (MS/MS). The liquid chromatography can be high-performance liquid chromatography (HPLC), also known as high pressure liquid chromatography, ultra performance (pressure) liquid chromatography (UPLC), or ultra-high performance (pressure) liquid chromatography (UHPLC).

### DEFINITIONS

By “subject” we mean an individual animal. The animal may be a mammal, including a human, a horse or a dog. The subject is typically, but not exclusively, involved in competitive and/or professional sports.

By “blood donation” we mean the collection of blood from the subject for storage and later re-infusion into the subject. We do not mean the taking of a small blood sample for testing purposes. Typically, “donation” applies to volumes of 50 mL-500 mL. A blood sample would more typically be a volume of approximately 50 μL-5 mL.

By “autologous blood transfusion” or “re-infusion” we mean the re-introduction of stored blood previously obtained from the subject back into the same subject.

By “blood sample” we include samples of blood including but not limited to whole blood samples, and isolated blood cells.

By “difference in level” we mean either an increased or decreased amount compared to the other sample, for example an increase in the amount of a particular peptide in the test sample compared to the reference sample; or alternatively a decrease in the amount of a particular peptide in the test sample compared to the reference sample. The amount of an increase can be can be more than 10%, such as more than 20%, 30%, 40%, 50%, such as preferably more than 60%, 70%, 80%, 90%, even more preferably more than 100%, 150%, or even more preferably than 200%. The amount of a decrease can be more than 10%, such as more than 20%, 30%, 40%, 50%, such as preferably more than 60%, 70%, 80%, 90%, up to an including a 100% decrease i.e. a complete absence of the peptide.

By “proteolytic peptide map” we mean the identification of the various peptides in a sample following proteolytic break down of proteins into such smaller peptides. The map typically takes the form of peptides being defined by mass and/or sequence. [Thiede et al. Peptide mass fingerprinting. Methods 2005. 35, 237-247].

### EXAMPLES

### Material & Methods

Subjects

In total, 14 individuals participated in the study. Seven healthy women and seven healthy men were recruited for donation, one unit for women and two units for men, where one unit corresponds to 450 mL blood.

Study Design

TABLE 1
Time line

| N = 7 Men; | Week | | | | | | | | | |
|---|---|---|---|---|---|---|---|---|---|---|
| 7 Women | 0 | 1 | 2 | 3 | 4 | 5 | 6 | 7 | 8 | 9 |

| | | | | | | | | | |
|---|---|---|---|---|---|---|---|---|---|
| VO_{2MAX} | T | | | | | | | | |
| Blood sampling | ▴ | ▴ | ▴ | | ▴●● | ● | ● | ● | ● |
| Blood Donation | | | D | D | | | | | |
| RBC re-infusion | | | | | R | | | | |

| State of | Clean | Donated | Doped |
|---|---|---|---|
| Transfusion group | | | |

Time lines showing blood donation and re-infusion, blood sampling and VO_{2MAX} testing for the discovery phase study. In total, 7 men and 7 women have participated, and 10 samples (▴ clean sample, ● doped sample) and 3 bag-samples were taken from each person. D; Donation of one unit (450 mL) blood (for women at week 3 only). R; Re-infusion of RBC. T; Testing of physical performance.

Blood Sampling, Donation and Transfusion

After an initial treadmill running test of VO_{2MAX}, one blood sample was taken each week for three weeks to establish individual baseline values. On week 2 and 3, one unit (450 mL) whole blood was donated by all men, and all women donated 450 mL on week 3 only. Blood sampling, donation, cryopreservation of donated blood and re-infusion was all performed at the Centre for Apheresis and stem cell handling, Karolinska University Hospital, Huddinge, Sweden. Blood donation followed standard hospital procedures in Sweden regarding illnesses, staying abroad, tattoos or drugs. Red blood cells were isolated and processed for cryopreservation as described elsewhere [Hutt et al. Transfusion of cryopreserved human red blood cells into healthy humans is associated with rapid extravascular hemolysis without a proinflammatory cytokine response. Transfusion. 2013; 53(1):28-33.) and RBCs stored at −80° C. for 3-4 weeks, unit one and two, respectively, before RBC re-infusion. Re-infusion of both units of washed RBC took place three weeks after donation of the first unit. Blood samples (1×4.5 mL EDTA) were taken.

Blood Collection

3.5 mL venous blood was drawn from each subject and collected in BD Vacationer Blood Collection Tubes containing EDTA (BD). Tubes were slowly turned 20 times and kept at room temperature for 30 minutes after which they were kept cold until further analysis.

Preparation of Erythrocytes from Fresh Blood

Lysis buffer (5 mM phosphate buffer with 1 mM EDTA, pH 7.6) and wash buffer (5 mM phosphate buffer with 1 mM EDTA and 150 mM NaCl, pH 7.6) were prepared one day before use. On day of use protein inhibitor tablets (Artnr. 05056489001, Roche Applied Science) were added to buffers (1 tablet/50 mL lysis buffer, 1 tablet/100 mL wash buffer). 35 mL cold wash buffer was added to centrifuge tubes (Beckman 50 mL). 1 mL blood was transferred to centrifuge tubes with buffer, mixed gently and centrifuged (Beckman Coulter, Avanti J-20 XP, JA17 or 25.50 rotor) at 1300×g, 8° C. for 10 min. The plasma and buffy coat fractions were carefully removed and discarded. 1.0 mL of the RBC fraction was transferred to clean centrifuge tubes and 35 mL cold ice wash buffer was added. Centrifuge tubes were gently shaken until the RBC's were dissolved. Samples were centrifuged at 8° C. and 1500×g, 10 min, with slow brake-settings (same centrifuge as above). 33 mL supernatant was discarded, 33 mL wash buffer added and the pellet was dissolved by gentle shaking. The centrifugation and cleaning steps were repeated twice. After the last centrifugation as much supernatant as possible (without disturbing the pellet) was removed.

Preparation of RBC Cytosol

RBC pellet was dissolved in 20 mL ice cold lysis buffer and shaken 30 min at −4° C. Samples were centrifuged at 25 000×g, 15 min, at 8° C. with slow brake-settings (Beckman Coulter, Avanti J-20 XP, JA17 or 25.50 rotor). 15 mL supernatant (collected from middle of fraction) was transferred to clean Centrifuge tubes. The centrifugation step was repeated once. 1.5 mL supernatant was aliquoted to four 2.0 mL micro tubes (2.0 mL Clear MAXYclear, Axygen).

Protein Concentration Measurements

All protein concentration measurements were performed using the Bradford assay. Coomassie Protein Assay reagent (Thermo Fischer) was prepared and used as described by the manufacturer's instructions (Thermo Fischer, art. no. #23200). Following a 15 min incubation the absorbance at 595 nm for bovine serum albumin standard samples (ranging from 100-600 ng/μl) and RBC cytosol samples were measured in triplicate on a Multiskan GO plate reader (Thermo Fischer). Concentration of samples was calculated from the linear fit of the standard curve.

Removal of Hemoglobin

Hemoglobin depletion was performed using HemoVoid resin, buffers and filter tubes as described by the manufacturer (Biotech Support Group) using a standard bench-top centrifuge. To filter tubes containing 15 mg HemoVoid resin 200 pl, HemoVoid binding buffer (HVBB) was added and tubes were mixed end-over-end for 5 min at room temperature. Tubes were centrifuged 5 min at 825×g to remove HVBB and the wash was thereafter repeated one more time. To filter tubes 300 μl HVBB was added together with 300 μl RBC cytosol sample and tubes were mixed end-over-end for 15 min at room temperature. Tubes were centrifuged 8 min in room temperature, at 6,000 rpm. The flow-through was discarded and sample loading was repeated one more time using 300 μl additional RBC cytosol sample in the same way. HemoVoid resin was washed with 500 μl HemoVoid wash buffer (HVWB) for 5 min at room temperature on an end-over-end rotator, followed by centrifugation as above and discarding of flow-through. The resin was similarly repeated two additional times before performing sample elution by addition of 150 μl HemoVoid elution buffer (HVEB), end-over-end mixing for 15 min at room temperature and finally centrifugation for 4 min in room temperature at 10,000 rpm.

Trypsin Digestion and Clean-Up

Equivalent amounts of samples were transferred to 0.8 mL 96-well format plates (Waters) for trypsin digestion and C18 cleanup. Assuming an average protein MW of 30 kDa for the hemoglobin depleted RBCc fractions, pre-digest internal peptide standards solubilized in Milli-Q™ grade (MQ)-water were added to samples at a final molar ratio of 1:100. Samples were then first reduced for 60 min at 60° C. in 5.1 mM dithiothreitol (Bio-Rad) and thereafter alkylated for 30 min at 22° C. by addition of iodoacetamide (Bio-Rad) to a final concentration of 25 mM. Trypsin Gold (Promega) was added to a final ratio of 0.54 U per pg protein sample (approximately a 1:30 (w/w) ratio) and samples were incubated for 16 h at 37° C. In order to stop the reaction and acidify samples (to a final pH of 3-4) for C18 binding acetonitrile and trifluoroacetic acid (TFA) were added to final concentrations of 5% (v/v) acetonitrile and 1% (v/v) TFA.

Clean-up of trypsin digest samples was performed in 96-well format at room temperature with a Positive Pressure 96 unit (Waters) using 10 mg bed weight Hypersep C18 plates (Thermo Fischer) according to the manufacturer's instructions. In brief, the resin was washed twice with 500 μl 50% (v/v) methanol and thereafter equilibrated twice with 5% (v/v), 0.5% (v/v) TFA. Samples were applied to the resin and the flow-through was after collection applied again to the resin. The resin was washed twice with 500 μl 5% (v/v), 0.5% (v/v) TFA and tryptic peptides were finally eluted into new 0.8 mL 96-well plates (Waters) in 100 μl 70% (v/v) acetonitrile. Acetonitrile was evaporated in a SpeedVac and dried samples were solubilized for 2 h at room temperature in 100 μl MQ-water before storage at −80° C.

Nano LC-MSMS Analysis

Samples to be analysed were transferred to LC vials (1 mL, TruView LCMS Certified Clear glass 12×32 mm screw neck total recovery vial with cap and preslit PTFE/silicone septa, Prod. No. 186005663CV, Waters). Samples used for analysis were thawed and diluted to the desired concentration with MQ-water. Internal retention time standards (iRT, Biognosys), pre-LC internal standard and TFA were added to final concentrations of 8 fmol/μl, 8 fmol/μl and 0.1% (v/v) respectively. 5 μL sample, corresponding to 50-400 ng peptide sample, was injected on a nanoACQUITY UltraPerformance UPLC™ (Waters), with full loop injection at a flow rate of 0.33 μL/min. Peptides were first bound to a nanoEase M/Z Symmetry C18 Trap Column (100Å, 5 μm, 180 μm×20mm, 2G, #186008821, Waters) and then separated on a nanoEase MZ HSS T3 column (15K psi, 100 Å, 1.8μm, 75 μm×250mm, #186008818, Waters) using a column temperature of 40° C. and sample temperature of 8° C. Inorganic phase solvent (solvent A) consisted of 0.1% formic acid (FA) in water, organic phase solvent (solvent B) of 99.9% acetonitrile (w/v), 0.1% (v/v) FA, seal wash of 10% (w/v) acetonitrile, weak wash of 1% (w/v) acetonitrile, 0.1% (v/v) TFA, lock spray solution of 25% (w/v) acetonitrile, 0.1% (v/v) FA, 0.5% (v/v) Leu-Enk, 1.6% (v/v) Glu-Fib. Following an 8.5 minute step at solvent ratio 95% A/5% B, samples were separated on a 60 min gradient from 95% A/5% B to 60% A/40% B. Thereafter a 1.5 min gradient to 15% A/85% B was applied and held for 2.5 minutes before the column was finally washed for 30 mins with 95% A/5% B. Separated peptides were analysed by mass spectrometry using a nano-UPLC Synapt G2Si™ (Waters). The mass analyser was run in positive resolution mode, analytes were ionized by electrospray ionization (ES) using a mass range of 50-2000 Da, scan time of 0.5 s, sampling cone voltage 30 V. Data acquisition and initial data evaluation were performed using MassLynx software (ver. SCN901, Waters).

Data Processing and Analysis

Raw data processing, alignment, peptide identification, quantification and comparative analysis were performed using Progenesis QI for Proteomics (v.3.0.6039.34628, Waters), Proteinlynx Global Server (ver. 3.0.3, Waters) and Skyline daily (ver. 3.7.1.11208). For peptide identification a curated human proteome database was used (Swiss-Prot), acquired from Uniprot (www.uniprot.org) (ver. 10th August 2016) in FASTA format using the following identification parameters: missed cleavages ≤2, variable modifications carbamidomethylation of cysteine, N-terminal acetylation, oxidation of methionine, peptide tolerance 10 ppm, fragment tolerance 25 ppm, false discovery rate <2%, fragments per peptide ≥1, fragments per protein ≥3. Common contaminants and internal standards were added to the database. Additional multivariate statistical analysis was performed using SIMCATM (Umetrics AB).

### Results

Identification of Biomarkers

The difference in the level of individual peptides in tryptic peptide maps in samples obtained from individuals having received autologous blood transfusion were compared to the level of the same specific peptides in reference tryptic peptide maps in samples obtained from a reference population of non-transfused control subjects.

Peptides with significant difference in levels are listed in Table 2 and the corresponding proteins are listed in Table 3.

These peptides, and the corresponding proteins, can be used as biomarkers in methods for the detection of autologous blood doping.

TABLE 2
Specific peptides

| Retention | Retention time | | Peptide Variable modifications | |
|---|---|---|---|---|
| time (min) | window (min) | Mass | ([position] description) | SEQ ID No. |
| 69.17 | 0.41 | 1541.82 | GAYIYNALIEFIR | 1 |
| 57.33 | 0.60 | 1583.73 | SIQFVDWCPTGFK | 2 |
| | | | [8] Carbamidomethyl C | |
| 48.99 | 0.37 | 1539.71 | WELNSGDGAFYGPK | 3 |
| 60.67 | 0.24 | 2669.33 | VNNVVWDLDRPLEEDCTLELLK | 4 |
| | | | [16] Carbamidomethyl C | |
| 36.00 | 0.30 | 751.30 | QAEEEF | 5 |
| 58.54 | 0.22 | 1805.96 | NAQLAQYNFILVVGEK | 6 |
| 45.52 | 0.31 | 2319.09 | AAAASAAEAGIATTGTEDSDDALLK | 7 |
| 37.98 | 0.46 | 1371.74 | AIADTGANVVVTGGK | 8 |
| 56.41 | 0.24 | 1695.83 | NSSYFVEWIPNNVK | 9 |
| 41.61 | 0.33 | 1157.58 | IDIDPEETVK | 10 |
| 22.40 | 0.29 | 1382.67 | DSSREASTSNSSR | 11 |
| 52.88 | 0.42 | 1142.62 | LAVNMVPFPR | 12 |
| 22.86 | 0.41 | 940.52 | TLPKSMHK | 13 |
| 60.07 | 0.36 | 1386.77 | ITVNEVELLVMK | 14 |
| 62.88 | 0.27 | 2408.18 | FDGALNVDLTEFQTNLVPYPR | 15 |
| 69.16 | 0.39 | 1598.84 | TPLLLMLGQEDRR | 16 |
| | | | [1] Acetyl N-terminal | |
| | | | [6] Oxidation M | |
| 28.57 | 0.33 | 1439.60 | SYNKDLESAEER | 17 |
| 37.47 | 0.34 | 1643.78 | ITCLCQVPQNAANR | 18 |
| | | | [3] Carbamidomethyl C | |
| | | | [5] Carbamidomethyl C | |
| 48.05 | 0.33 | 1323.77 | AVAVVVDPIQSVK | 19 |
| 41.78 | 0.24 | 2156.08 | TVGTPIASVPGSTNTGTVPGSEK | 20 |
| 37.93 | 0.47 | 1465.68 | QDEWIKFDDDK | 21 |
| | | | [1] Methylation | |
| | | | [6] Methylation | |
| 41.59 | 0.63 | 1615.79 | QPAENVNQYLTDPK | 22 |
| 42.62 | 0.45 | 812.51 | NIILGGVK | 23 |
| 36.51 | 0.85 | 1664.74 | DTSQSDKDLDDALDK | 24 |
| 36.51 | 0.53 | 1664.74 | DTSQSDKDLDDALDK | 24 |
| 64.39 | 0.36 | 1597.91 | VWINTSDIILVGLR | 25 |
| 22.50 | 0.31 | 837.36 | YNADEAR | 26 |
| 22.76 | 0.41 | 1408.61 | EFQSPDEEMKK | 27 |
| | | | [1] Acetyl N-terminal | |
| 42.00 | 0.73 | 730.40 | DAAITLK | 28 |
| 39.33 | 0.43 | 1496.77 | QTLQSEQPLQVAR | 29 |
| 61.66 | 0.66 | 2023.10 | DTYARWLPLGLGLNHLGK | 30 |
| 33.77 | 0.27 | 659.34 | VAEWR | 31 |
| 43.88 | 0.58 | 1867.90 | LALCLCMINFYHGGLK | 32 |
| | | | [6]Carbamidomethyl C | |
| | | | [7] Oxidation M | |
| 33.77 | 0.41 | 730.38 | VAAEWR | 33 |
| 40.96 | 0.30 | 1587.75 | AVEAAELCLEQNNK | 34 |
| | | | [8] Carbamidomethyl C | |
| 62.70 | 0.85 | 1248.58 | MEGPLSVFGDR | 35 |
| | | | [1] Acetyl N-terminal | |
| 45.79 | 0.41 | 1407.67 | AVFDETYPDPVR | 36 |
| 23.22 | 0.41 | 1564.64 | AAEDDEDDDVDTKK | 37 |
| 32.01 | 0.61 | 1120.54 | GYLPSHYER | 38 |
| 29.73 | 0.31 | 740.38 | EFFNGK | 39 |
| 53.60 | 0.29 | 1606.81 | GFQEVVTPNIFNSR | 40 |
| 31.83 | 0.53 | 1479.62 | QCNRHYCWEK | 41 |
| | | | [2] Carbamidomethyl C | |
| | | | [7] Carbamidomethyl C | |
| 22.48 | 0.38 | 1223.65 | VAAAETAKHQAK | 42 |
| 36.27 | 0.15 | 1498.76 | ARDAAEFELFFR | 43 |
| | | | [2] Methylation | |
| | | | [3] Methylation | |
| 33.79 | 0.26 | 931.46 | QDADSLQR | 44 |
| 51.79 | 0.42 | 1135.59 | LGIRFCTNR | 45 |
| | | | [6] Carbamidomethyl C | |
| 23.22 | 0.38 | 1564.63 | AAEDDEDDDVDTKK | 37 |
| 22.76 | 0.34 | 1465.63 | ETDSSSASAATPSKK | 46 |
| 28.91 | 0.46 | 1407.61 | SNTAGSQSQVETEA | 47 |
| 38.30 | 0.29 | 1278.61 | EDGQEYAQVIK | 48 |
| 23.22 | 0.43 | 1621.66 | RMTGSEFDFEEMK | 49 |
| | | | [12] Oxidation M | |
| 23.61 | 0.34 | 1092.46 | MGHFTEEDK | 50 |
| 22.21 | 0.26 | 1165.55 | NEQESAVHPR | 51 |
| 34.01 | 0.46 | 1733.85 | TQTPPVSPAPQPTEER | 52 |
| 40.62 | 0.22 | 628.39 | LLDLR | 53 |
| 30.39 | 0.41 | 959.47 | HLTGEFEK | 54 |
| 59.88 | 0.44 | 2320.15 | KYFHAQLQLEQLQEENFR | 55 |
| 27.65 | 0.24 | 699 35 | SMHLGR | 56 |
| 33.77 | 0.21 | 659.34 | VAEWR | 31 |
| 27.05 | 0.44 | 1288.62 | QLEDELAAMQK | 57 |
| | | | [10] Methylation | |
| 33.22 | 0.34 | 1964.86 | CGNCGPGYSTPLEAMKGPR | 58 |
| | | | [1] Methylation | |
| | | | [13] Methylation | |
| 23.23 | 0.34 | 1621.65 | RMTGSEFDFEEMK | 49 |
| | | | [12] Oxidation M | |
| 49.06 | 0.33 | 936.45 | SVQTFADK | 59 |
| | | | [1] Acetyl N-terminal | |
| 39.69 | 0.24 | 820.43 | FVVDVDK | 60 |
| 23.47 | 0.41 | 1186.60 | REQQPSVTSR | 61 |
| 28.99 | 0.46 | 1149.48 | MNPNCARCGK | 62 |
| | | | [5] Carbamidomethyl C | |
| 46.26 | 0.31 | 1919.95 | QVMVVPVGPTCDEYAQK | 63 |
| | | | [11] Carbamidomethyl C | |
| 44.61 | 0.41 | 1848.88 | QYTSPEEIDAQLQAEK | 64 |
| 34.49 | 0.22 | 898.44 | VLEAGDAQP | 65 |
| 36.46 | 0.26 | 1770.78 | AEEADHEVLDQKEMK | 66 |
| 32.78 | 0.49 | 1964.86 | MCKQDPSVLHTEEMR | 67 |
| | | | [1] Glycation N-Terminal | |
| 39.60 | 0.27 | 1551.69 | YDSNSGGEREIQR | 68 |
| | | | [1] Acetyl N-terminal | |
| 46.49 | 0.33 | 1894.94 | ELVFKEDGQEYAQVIK | 69 |
| 54.36 | 0.26 | 3047.48 | SLGSLPGSVVEANPNQRDPPLWDEIDSR | 70 |
| 61.09 | 0.21 | 2466.09 | MWDVSTGMCLMTLVGHDNWVR | 71 |
| | | | [8] Oxidation M | |
| 25.40 | 0.22 | 1024.52 | VAEIEHAEK | 72 |
| 33.53 | 0.27 | 1883.84 | GDYHRYLAEFATGNDR | 73 |
| 64.80 | 0.39 | 2460.13 | DQELYFFHELSPGSCFFLPK | 74 |
| | | | [15] Carbamidomethyl C | |
| 37.94 | 0.56 | 1228.64 | IHPTSVISGYR | 75 |
| 45.31 | 0.60 | 869.53 | LAVEAVLR | 76 |
| 49.01 | 0.39 | 940.47 | SLDNFFAK | 77 |
| 59.70 | 0.37 | 1309.76 | GGPNIITLADIVK | 78 |

TABLE 3
Proteins corresponding to the specific peptides identified in Table 2

| Peptide Variable modifications | SEQ | | |
|---|---|---|---|
| ([position] description) | ID No. | Protein | Description |
| GAYIYNALIEFIR | 1 | P26639\|SYTC_HUMAN | Threonine-tRNA ligase, cytoplasmic |
| SIQFVDWCPTGFK | 2 | P68363\|TBA1B_HUMAN | Tubulin alpha-1B chain |
| [8] Carbamidomethyl C | | | |
| WELNSGDGAFYGPK | 3 | P26639\|SYTC_HUMAN | Threonine-tRNA ligase, cytoplasmic |
| VNNVVWDLDRPLEEDCTLELLK | 4 | P26639\|SYTC_HUMAN | Threonine-tRNA ligase, cytoplasmic |
| [16] Carbamidomethyl C | | | |
| QAEEEF | 5 | P26639\|SYTC_HUMAN | Threonine-tRNA ligase, cytoplasmic |
| NAQLAQYNFILVVGEK | 6 | P26639\|SYTC_HUMAN | Threonine-tRNA ligase, cytoplasmic |
| AAAASAAEAGIATTGTEDSDDALLK | 7 | P55036\|PSMD4_HUMAN | 26S proteasome non-ATPase regulatory |
| | | | subunit 4 |
| AIADTGANVVVTGGK | 8 | P50990- | Isoform 2 of T-complex protein 1 |
| | | 2\|TCPQ_HUMAN | subunit theta |
| NSSYFVEWIPNNVK | 9 | P68371\|TBB4B_HUMAN | Tubulin beta-4B chain |
| IDIDPEETVK | 10 | P54727\|RD23B_HUMAN | UV excision repair protein RAD23 |
| | | | homolog B |
| DSSREASTSNSSR | 11 | P78318\|IGBP1_HUMAN | Immunoglobulin-binding protein 1 |
| LAVNMVPFPR | 12 | P68371\|TBB4B_HUMAN | Tubulin beta-4B chain |
| TLPKSMHK | 13 | Q96NW7- | Isoform 2 of Leucine-rich repeat- |
| | | 2\|LRRC7_HUMAN | containing protein 7 |
| ITVNEVELLVMK | 14 | Q9UNM6\|PSD13_HUMAN | 26S proteasome non-ATPase regulatory |
| | | | subunit 13 |
| FDGALNVDLTEFQTNLVPYPR | 15 | P68363\|TBA1B_HUMAN | Tubulin alpha-1B chain |
| TPLLLMLGQEDRR | 16 | P13798\|ACPH_HUMAN | Acylamino-acid-releasing enzyme |
| [1] Acetyl N-terminal | | | |
| [6] Oxidation M | | | |
| SYNKDLESAEER | 17 | O43242\|PSMD3_HUMAN | 26S proteasome non-ATPase regulatory |
| | | | subuni 3 |
| ITCLCQVPQNAANR | 18 | P49558\|SYAC_HUMAN | Alanine-tRNA ligase, cytoplasmic |
| [3] Carbamidomethyl C | | | |
| [5] Carbamidomethyl C | | | |
| AVAVVVDPIQSVK | 19 | O00487\|PSDE_HUMAN | 26S proteasome non-ATPase regulatory |
| | | | subunit 14 |
| TVGTPIASVPGSTNTGTVPGSEK | 20 | Q99460- | Isoform 2 of 26S proteasome non-ATPase |
| | | 2\|PSMD1_HUMAN | regulatory subunit 1 |
| QDEWIKFDDDK | 21 | P54579\|UBP14_HUMAN | Ubiquitin carboxyl-terminal hydrolase 14 |
| [1] Methylation | | | |
| [6] Methylation | | | |
| QPAENVNQYLTDPK | 22 | P22314- | Isoform 2 of Uniquitin-like modifier- |
| | | 2\|UBA1_HUMAN | activating enzyme 1 |
| NIILGGVK | 23 | P22314\|UBA1_HUMAN | Ubiquitin-like modifier-activating |
| | | | enzyme 1 |
| DTSQSDKDLDDALDK | 24 | P20810\|ICAL_HUMAN | Isoform 3 of Calpastatin |
| VWINTSDIILVGLR | 25 | O14602\|IF1AY_HUMAN | Eukaryotic translation initiation factor |
| | | | 1A, Y-chromosomal |
| YNADEAR | 26 | O14602\|IF1AY_HUMAN | Eukaryotic translation initiation factor |
| | | | 1A, Y-chromosomal |
| EFQSPDEEMKK | 27 | O75533\|SF3B1_HUMAN | Splicing factor 3B subunit 1 |
| [1] Acetyl N-terminal | | | |
| DAAITLK | 28 | Q07960\|RHG01_HUMAN | Rho GTPase-activating protein 1 |
| QTLQSEQPLQVAR | 29 | P35998\|PRS7_HUMAN | 26S protease regulatory subunit 7 |
| DTYARWLPLGLGLNHLGK | 30 | Q13200\|PSMD2_HUMAN | 26S protease non-ATPase regulatory |
| | | | subunit 2 |
| VAEWR | 31 | Q9Y230\|RUVB2_HUMAN | RubB-like 2 |
| LALCLCMINFYGHHLK | 32 | Q15042\|RB3BP_HUMAN | Rab3 GTPase-activating protein catalytic |
| [6] Carbamidomethyl C | | | subunit |
| [7] Oxidation M | | | |
| VAAEWR | 33 | Q02218- | Isoform 2 of 2-oxoglutarate |
| | | 2\|ODO1_HUMAN | dehydrogenase, mitochondrial |
| AVEAAELCLEQNNK | 34 | P10746\|HEM4_HUMAN | Uroporphyrinogen-III synthase |
| [8] Carbamidomethyl C | | | |
| MEGPLSVFGDR | 35 | P17987\|TCPA_HUMAN | T-complex protein 1 subunit alpha |
| [1] Acetyl N-terminal | | | |
| AVFDETYPDPVR | 36 | P49588\|SYAC_HUMAN | Alanine-tRNA ligase, cytoplasmic |
| AAEDDEDDDVDTKK | 37 | P06454- | Isoform 2 of Prothymosin alpha |
| | | 2\|PTMA_HUMAN | |
| GYLPSHYER | 38 | Q6P2Q9\|PRP8_HUMAN | Pre-mRNA-processing-splicing factor 8 |
| EFFNGK | 39 | P11021\|GRP78_HUMAN | 78 kDa glucose-regulated protein |
| GFQEVVTPNIFNSR | 40 | P26639\|SYTC_HUMAN | Threonine-tRNA ligase, cytoplasmic |
| QCNRHYCWEK | 41 | Q9P0U4- | Isoform 2 of CXXC-type zinc finer |
| [2] Carbamidomethyl C | | 2\|CXXC1_HUMAN | protein 1 |
| [7] Carbamidomethyl C | | | |
| VAAAETAKHQAK | 42 | P11532\|DMD_HUMAN | Dystrophin |
| ARDAAEFELFFR | 43 | Q6XQN6\|PNCB\|HUMAN | Nicotinate phosphoribosyltransferase |
| [2] Methylation | | | |
| [3] Methylation | | | |
| QDADSLQR | 44 | P14324\|FPPS_HUMAN | Farnesyl pyrophosphate synthase |
| LGIRFCTNR | 45 | [13798\|ACPH_HUMAN | Acylamino-acid-releasing enzyme 4 |
| [6] Carbamidomethyl C | | | |
| ETDSSSASAATPSKK | 46 | P54578- | Isoform 2 of Ubiquitin carboxy-terminal |
| | | 2\|UBP14_HUMAN | hydrolase 14 |
| SNTASGSQSQVETEA | 47 | Q02790\|FKVP4_HUMAN | Peptidyl-prolyl cis-trans isomerase |
| | | | FKBP4 |
| EDGQEYAQVIK | 48 | O14602\|IF1AY_HUMAN | Eukaryotic translation initiation factor |
| | | | 1A, Y-chromosomal |
| RMTGSEFDFEEMK | 49 | P50396\|GDIB_HUMAN | Rab GDP dissociation inhibitor beta |
| [12] Oxidation M | | | |
| MGHFTEEDK | 50 | P69891\|HBG1_HUMAN | Hemoglobin subunit gamma-1 |
| NEQESAVHPR | 51 | Q9UJU6\|DBNL_HUMAN | Drebrin-like protein |
| TQTPPVSPAPQPTEER | 52 | Q14247\|SRC8_HUMAN | Src substrate cortactin |
| LLDLR | 53 | Q8IZT6\|ASPM_HUMAN | Abnormal spindle-like mircocephaly- |
| | | | associated protein |
| HLTGRFEK | 54 | P62826\|RAN_HUMAN | GTP-binding nuclear protein Ran |
| KYFHAQLQLEQLQEENFR | 55 | Q9UJC3\|HOOK1_HUMAN | Protein Hook homolog 1 |
| SMHLGR | 56 | Q9Y570\|PPME1_HUMAN | Protein phosphatase methyltransferase 1 |
| QLEDEALAAMQK | 57 | P06753\|TPM3_HUMAN | Tropomyosin alpha-3 chain |
| [10] Methylation | | | |
| [1] Methylation | 58 | Q13228\|SBP1_HUMAN | Selenium-binding protein 1 |
| [13] Methylation | | | |
| SVQTFADK | 58 | P47756- | Isoform 2 of F-actin-capping protein |
| [1] Acetyl N-terminal | | 2\|CAPZB_HUMAN | subunit beta |
| FVVDVDK | 60 | P62195\|PRS8_HUMAN | Isoform 2 of 26S protease regulatory |
| | | | subunit 8 |
| REQQPSVTSR | 61 | P25686- | Isoform2 of DanJ homolog subfamily B |
| | | 2\|DNJB2_HUMAN | member 2 |
| MNPNCARCGK | 62 | Q14847\|LASP1_HUMAN | LIM and SH3 domain protein 1 |
| [5] Carbamidomethyl C | | | |
| QVMVVPVGPTCDEYAQK | 63 | P26639\|SYTC_HUMAN | Threonine-tRNA ligase, cytoplasmic |
| [11] Carbamidomethyl C | | | |
| QYTSPEEIDAQLQAEK | 64 | Q13442\|HAP28_HUMAN | 28 kDa heat- and acid-stable |
| | | | phosphoprotein |
| VLEAGDAQP | 65 | Q5JRK9\|GGEE3_HUMAN | Putative G antigen family E member 3 |
| AEEADHEVLDQKEMK | 66 | Q`4789- | Isoform 4 of Golgin subfamily B member 1 |
| | | 4\|GOGB1_HUMAN | |
| MCKQDPSVLHTEEMR | 67 | Q8NFI4\|F10A5_HUMAN | Putative protein FAM10A5 1 |
| [1] Glycation N-terminal | | | |
| YDSNSGGEREIQR | 68 | P62191\|PRS4_HUMAN | 26S protease regulatory subunit 4 |
| [1] Acetyl N-terminal | | | |
| ELVFKEDGQEYAQVIK | 69 | O14602\|IF1AY_HUMAN | Eukaryotic translation initiation factor |
| | | | 1A, Y-chromosomal |
| SLGSLPGSVVEANPNQRDPPLWDEIDSR | 70 | Q16864- | Isoform 2 of V-type proton ATPase |
| | | 2\|VATE_HUMAN | subunit F |
| MWDVSTGMCLMTLVGHDNWVR | 71 | P43043\|LIS1_HUMAN | Platelet-activating factor acetyl- |
| [8] Oxidation M | | | hydrolase IB subunit alpha |
| VAEIEHAEK | 72 | P78371\|TCPB_HUMAN | T-complex protein 1 subunit beta |
| GDYHRYLAEFATGNDR | 73 | P62258- | Isoform SV of 14-3-3 protein epsilon |
| | | 2\|1433E_HUMAN | |
| DQELYFFHELSPGSCFFLPK | 74 | P26639\|SYTC_HUMAN | Threonine-tRNA ligase, cytoplasmic |
| [15] Carbamidomethyl C | | | |
| IHPTSVISGYR | 75 | P17987\|TCPA_HUMAN | T-complex protein 1 subunit alpha |
| LAVEAVLR | 76 | P78371\|TCPB_HUMAN | T-complex protein 1 subunit beta |
| SLDNFFAK | 77 | Q9UKY7\|CDV3_HUMAN | Protein CDV3 homolog |
| GGPNIITLADIVK | 78 | P68400\|CSK21_HUMAN | Casein kinase II subunit alpha |

1. A method for detection of autologous blood-doping with cryopreserved blood in a subject, said method comprising the step:
i) identifying whether said subject has or has not been autologous blood doped based on differences in level of one or more specific peptides between an isolated red blood cell cytosol sample prepared from a blood sample from said subject compared to the level of the same specific peptides from an isolated red blood cell cytosol sample prepared from a reference blood sample, such levels having been determined by generation of a proteolytic peptide map for each of the blood sample and the reference blood sample.

2. A method as claimed in claim 1 further comprising performing before step (i) one or more of the steps of:
x) determining the level of one or more specific peptide in the isolated red blood cell cytosol sample prepared from the blood sample obtained from said subject;
y) determining the level of one or more specific peptide in an isolated red blood cell cytosol sample prepared from a reference blood sample;
z) determining any difference in level of said one or more specific peptides compared to the level of the same specific peptides in a reference blood sample.

3. A method as claimed in either of claim 1 or 2, said method comprising the steps:
a) generating a proteolytic peptide map of an isolated red blood cell cytosol sample prepared from a blood sample obtained from said subject;
b) determining the levels of one or more specific peptide identified in the peptide map obtained in step a)
c) determining the difference in level of said one or more specific peptides compared to the level of the same specific peptides in reference peptide maps obtained from an isolated red blood cell cytosol sample prepared from a reference blood sample; and
d) identifying whether said subject has or has not been autologous blood doped with cryopreserved blood based on differences in the level of said specific peptides compared to the level of the same specific peptides in said reference peptide maps.

4. A method as claimed in any previous claim wherein the reference blood sample is from a non-doped subject.

5. The method according to any previous claim wherein said one or more specific peptides is or are one or more peptides derived from one or more of the proteins listed in Table 3.

6. The method according to any previous claim wherein said one or more specific peptides is or are one or more peptides selected from the list of peptides comprising SEQ ID Nos: 1-78.

7. The method according to any previous claim wherein said one or more specific peptides is or are 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, or 78 peptides selected from the list of peptides comprising SEQ ID Nos: 1-78.

8. The method according to any previous claim wherein said one or more specific peptides is or are one or more peptides derived from one or more of the proteins selected from: Threonine-tRNA ligase, cytoplasmic; 26S proteasome non-ATPase regulatory subunit 4; Isoform 2 of T-complex protein 1 subunit theta; Tubulin beta-4B chain UV excision repair protein RAD23 homolog B; 26S proteasome non-ATPase regulatory subunit 13; Isoform 2 of Ubiquitin-like modifier-activating enzyme 1; Isoform 3 of Calpastatin; Uroporphyrinogen-III synthase; and Casein kinase II subunit alpha.

9. The method according to any previous claim wherein said one or more specific peptides is or are one or more peptides consisting of the amino acid sequence of one of the peptides SEQ ID Nos: 1, 7, 8, 9, 10, 12, 14, 22, 24, 34, and 78.

10. The method according to any previous claims wherein the step of generating the proteolytic peptide map comprises protease digestion that is performed using one or more of the proteases selected from trypsin, chymotrypsin, Lys-C, Gly-C, Asp-N, Arg-C, papain.

11. The method according to claim 10 wherein the protease digestion is performed by trypsin digestion.

12. The method according to any previous claim wherein the peptide mapping is performed by the combination of liquid chromatography (LC) with mass spectrometry (MS), and preferably wherein the MS is tandem mass spectrometry (MS/MS).

13. Use of a peptide selected from the list of peptides comprising the peptides SEQ ID Nos: 1-78 in a method as described in any one of the preceding claims for detection of autologous blood-doping with cryopreserved blood.

14. A peptide consisting of the amino acid sequence of one the peptides SEQ ID Nos:1, 7, 8, 14, and 34.

15. A kit comprising one or more of the peptides according to claim 14.

16. A method for the identification of biomarkers for the detection of autologous blood-doping with cryopreserved blood, said method comprising the steps;
i) identifying differences in the level of one or more peptides between an isolated red blood cell cytosol sample prepared from blood samples obtained from one or more subjects having received an infusion of autologous blood and an isolated red blood cell cytosol sample prepared from blood samples obtained from one or more control subjects not having received an infusion of autologous blood, such levels having been identified following generation a proteolytic peptide map of the blood samples; and
ii) identifying peptides being present in significant different levels, as a biomarker for autologous blood doping with cryopreserved blood.

17. A method as claimed in claim 16, said method comprising the steps;
i) generating proteolytic peptide maps of an isolated red blood cell cytosol sample prepared from blood samples obtained from one or more individuals having received an infusion of autologous blood,
ii) generating proteolytic peptide maps of an isolated red blood cell cytosol sample prepared from blood samples obtained from one or more control individuals not having received an infusion of autologous blood,
iii) identifying differences in the level of one or more peptides in the peptide maps obtained in step i) compared to peptide maps obtained in step ii), and
iv) identifying peptides being present in significant different levels, and the corresponding proteins, as a biomarker for autologous blood doping with cryopreserved blood.

18. The method according to claim 16 or 17, wherein said cytosol is depleted of hemoglobin or has a reduced level of hemoglobin.
